(19) United States
(12) Patent Application Publication
(10) Pub. No.: US 2025/0352760 A1
(43) Pub. Date: Nov. 20, 2025

(54) Medical dispensing system with self-closing slit valves

(71) Applicant: Heraeus Medical GmbH, Wehrheim (DE)

(72) Inventors: Sebastian VOGT, Wehrheim (DE); Moritz WARMBIER, Wehrheim (DE); Thomas KLUGE, Wehrheim (DE); Roland STANGL, Moosburg (DE)

(21) Appl. No.: 19/205,462

(22) Filed: May 12, 2025

(30) Foreign Application Priority Data
May 17, 2024 (EP) 24176560.1

(51) Int. Cl. A61M 25/00; A61M 25/01; A61M 39/24

(52) U.S. Cl. CPC A61M 25/0045; A61M 25/0015; A61M 25/005; A61M 25/0068; A61M 25/0108; A61M 39/24; A61M 2202/0007; A61M 2202/04; A61M 2205/0216; A61M 2205/32; A61M 2207/00

(57) ABSTRACT

A device for dispensing a medical fluid, comprising a tube comprising a first medically acceptable material with a Shore A hardness in the range of 75 to 95, wherein the tube has an inner diameter (ID) and an outer diameter (AD), wherein the ratio of [ID:AD] is in the range of [1:1.8] to [1:2.5]; wherein the tube comprises a first end for receiving a fluid into the tube; wherein the tube comprises a second end configured to retain a fluid in the tube; one or more slits for dispensing a fluid from the tube, wherein the slits each form a passage extending from an inner side of the tube to an outer side of the tube; and wherein the slits are configured to reversibly open depending on the pressure of a fluid within the tube so that the fluid is dispensed from the slits.

### CROSS-REFERENCE TO RELATED APPLICATION

This application claims priority pursuant to 35 U.S.C. 119 (a) to European Patent Application No. 24176560.1, filed May 17, 2024, which application is incorporated herein by reference in its entirety.

### FIELD OF THE INVENTION

The present application relates to a device for the temporary, local application of medical fluids over a period of hours to several days. Depending on the anatomical situation of the implantation site, the device according to the invention can be adapted in terms of its length by simple mechanical shortening without any loss of function.

### BACKGROUND OF THE INVENTION

The local application of pharmaceutical active ingredients, in particular antibiotics, is generally known and has proven in particular effective in the treatment or calming of infections of the bone tissue. A distinction can be made between non-absorbable and absorbable or biodegradable active ingredient carriers.

Examples of non-absorbable active ingredient carriers are the bead chains known since 1977 under the brand name Septopal®. These consist of polymethyl methacrylate beads containing the broad-spectrum antibiotic gentamicin sulfate, with these beads arranged in a chain on steel thread. This active ingredient carrier in chain form has proven effective for decades in the local antibiotic treatment of osteomyelitis. The advantage here is that the gentamicin sulfate is released from the active ingredient carrier in larger quantities over a period of several days. Another advantage is that the active ingredient carrier in chain form can be easily adapted to the anatomical situation at the implantation site by the medical user by simply cutting off excess beads. The disadvantage is that the active ingredient carrier contains only gentamicin sulfate and that the medical user cannot modify the active ingredient carrier with additional antibiotics according to the sensitivity of the microbial germs. As a result, successful local treatment of infections with problem germs such as MRSA and VRSA is only possible to a limited extent or not at all. The removal of the Septopal® chains after the active ingredient has been released is associated with considerable stress for the patient due to adhesion to connective tissue.

Examples of absorbable or biodegradable active ingredient carriers are fleeces and sponges made of collagen or gelatin. Examples of this are the patents DE3429038, DE3334595, DE2843963, DE3203957 and DE3334595. These contain gentamicin sulfate or mixtures of gentamicin sulfate and a gentamicin salt that is slightly soluble in water. Furthermore, there are a variety of absorbable or biodegradable active ingredient carriers based on tricalcium phosphate, hydroxyapatite, gypsum and their mixtures as well as composite materials made of these salts and organic binders.

The disadvantage of the listed non-absorbable and also the absorbable or biodegradable active ingredient carriers is that the antimicrobial active ingredient is determined by the selected composition and, in addition, that after the implantation of the active ingredient carrier, the active ingredient can no longer be replaced or supplemented with other active ingredients. Furthermore, the release of active ingredients in all previous local active ingredient release systems is based on the principle of diffusion, so that high amounts of active ingredients are only released in the first few hours and days. An exception is the use of active ingredient salts that are slightly soluble in water, where the release of the active ingredient depends on the solubility equilibrium of the active ingredient salts.

Therefore, an active ingredient carrier is desirable, which allows local application of any pharmaceutical active ingredient, and it being possible for the pharmaceutical active ingredient to be exchanged for other fluid pharmaceutical active ingredients at any time. In addition, it is desirable that the active ingredient concentration achieved directly at the implantation site can be directly adjusted.

EP3795196B1 discloses a device for the local application of a medical fluid, comprising a tube which is flexibly deformable and which comprises a tube wall, wherein the tube wall comprises a radially outer outer wall made of a first material, and the tube wall comprises a radially inner inner wall made of a second material, which delimits an inner line of the tube, wherein the tube comprises a plurality of openings in the tube wall in a distal section of the tube, wherein the plurality of openings connect the inner line of the tube to the surroundings of the tube, wherein the distal section of the tube is delimited by a distal end of the tube, the device further comprising a closure element with which the tube is or can be closed liquid-tightly at the distal end of the tube, wherein the closure element can be manually inserted into the distal end of the tube, wherein a proximal end of the tube is or can be connected to a container for the medical fluid in a liquid-permeable manner such that the medical fluid can be pressed out from the container through the proximal end of the tube into the inner line of the tube and can be pressed out through the plurality of openings to the surroundings of the tube.

EP3854437B1 describes a device for the local application of a medical fluid comprising a tube, wherein the tube comprises a plurality of openings in its tube wall which connect an inner line of the tube with the surroundings of the tube, and wherein the tube is closed at a distal tube end of the tube, wherein the device comprises an outer sleeve for fluid-tightly closing a portion of the plurality of openings, wherein the outer sleeve is arranged axially displaceably around the tube, and wherein the outer sleeve is shorter than the tube, so that the distal openings not belonging to the closed portion of the plurality of openings are exposed.

### SUMMARY OF THE INVENTION

An object of the present invention is to solve one or more of the problems described above and further problems of the prior art. An object of the invention is to provide a simple, cost-effective device for local active ingredient release. In some embodiments, a low-cost homogeneous tube can be used, thus avoiding the use of a costly coaxial tube made from two separate polymers. Such a device can allow for local application of medical fluids of any composition, for example antibiotic solutions. For this purpose, one part of the device can be placed inside the patient after implantation and a second part of the device can be placed outside the patient. The medical fluids can be introduced into the part of the device located outside the patient and passed through the device to the implantation site and released there. The device can be plastically deformed in order to be able to follow the anatomical conditions at the implantation site. The release of medical fluids can occur from openings arranged longitudinally on the device. The openings can be reversibly closable to prevent ingrowth of connective tissue and/or blockage of the openings by, for example, coagulated blood. It is desirable that the device does not expand radially during use or only expands radially to a minimal extent. Inflamed human tissue is very sensitive to pain under pressure. It is therefore preferable to avoid a significant radial extension of the device, which would cause pressure and thus pain for the patient. Another advantage is that repeated release of the medical liquid by applying pressure does not result in the openings tearing open or any other irreversible enlargement of the openings. Preferably, the opening characteristics of the openings of the device can be kept constant in order to ensure a uniform release of the pharmaceutical liquid over the entire length of the device even in the event of multiple liquid releases. Furthermore, the device can be designed in such a way that the part of the device that can be positioned in the patient can be adapted to the relevant anatomical situation of the patient by shortening the length of a tube, without impairing the function of the device.

These objects are achieved by the methods, devices, kits and medical uses described herein, in particular those which are described in the claims.

Preferred embodiments of the invention will be described below.

A first embodiment of the invention is a device for dispensing a medical fluid to a patient, comprising a tube comprising a first medically acceptable material with a Shore A hardness in the range of 75 to 95, wherein the tube has an inner diameter (ID) and an outer diameter (AD), wherein the ratio of [ID:AD] is in the range of [1:1.8] to [1:2.5]; wherein the tube comprises a first end for receiving a fluid into the tube; wherein the tube comprises a second end configured to retain a fluid in the tube; one or more slits for dispensing a fluid from the tube, wherein the slits each form a passage extending from an inner side of the tube to an outer side of the tube; and wherein the slits are configured to reversibly open depending on the pressure of a fluid within the tube so that the fluid is dispensed from the slits.

A second embodiment relates to a device according to the first embodiment, wherein the device is designed and configured to dispense a medical fluid exclusively via the slits, wherein preferably a simultaneous dispensation of the fluid takes place via a plurality of slits at different positions along the longitudinal axis of the tube.

A third embodiment relates to a device according to the first or second embodiment, wherein the slits are designed and configured to open above a limit pressure of a fluid inside the tube, which is 1 bar (10\hat{ }5 Pa), preferably 1.4 bar, higher than the pressure outside the tube, and to close fluid-tight below this limit pressure.

A fourth embodiment relates to a device according to one of the preceding embodiments, wherein the slits are configured to reversibly open and close via a resilient restoring force of the first material of the tube.

A fifth embodiment relates to a device according to one of the preceding embodiments, wherein the slits each have a slit length L, and wherein the ratio of the slit length (L) to the inner diameter (ID) of the tube [L:ID] is in the range from [1:2.2] to [1:2.9], and/or the ratio of the slit length L to the outer diameter AD of the tube [L:AD] is in the range from [1:3.5] to [1:5.5].

A sixth embodiment relates to a device according to one of the preceding embodiments, wherein the device is designed and configured to change the outer diameter AD of the tube by less than 10% when the pressure of a fluid inside the tube increases from 0 to 1 bar (10\hat{ }5 Pa) higher than the pressure outside the tube.

A seventh embodiment relates to a device according to one of the preceding embodiments, wherein the device is designed and configured to change the length of the tube by less than 10%, preferably less than 5%, when the pressure of a fluid inside the tube increases from 0 to 1 bar (10\hat{ }5 Pa) higher than the pressure outside the tube.

An eighth embodiment relates to a device according to one of the preceding embodiments, wherein the tube comprises a plurality of slits, each arranged at a distance (A) from one another on the outer side of the tube, and each having a slit length (L) along the outer side of the tube, wherein the ratio of distance (A) to slit length (L) [A:L] is at least [10:1].

A ninth embodiment relates to a device according to one of the preceding embodiments, wherein the tube comprises a second material which has a higher Shore A hardness than the first material, wherein the second material is preferably arranged as a coaxially encircling layer or as a strip parallel to a longitudinal axis of the tube, wherein the second material further preferably comprises an elastomer, and wherein further preferably the second material is completely embedded in the first material.

A tenth embodiment relates to a device according to the ninth embodiment, wherein the second material comprises a radiopaque agent and/or a dye, wherein the radiopaque agent preferably comprises barium sulfate or tungsten, and wherein the dye preferably has an absolute emission maximum in the range of 490 nm to 575 nm.

An eleventh embodiment relates to a device according to one of the preceding embodiments, wherein the slits are arranged parallel to a longitudinal axis of the tube.

A twelfth embodiment relates to a device according to one of the preceding embodiments, wherein in a first longitudinal position in the direction of the longitudinal axis of the tube a first slit is arranged at a first radial position, and in a second longitudinal position in the direction of the longitudinal axis of the tube a second slit is arranged at a second radial position, wherein the first radial position forms an angle of approximately 90° to the second radial position.

A thirteenth embodiment relates to a device according to one of the preceding embodiments, wherein the tube comprises a support structure made of metal, wherein the support structure preferably comprises a metal coating, a metal foil, a metal spiral or a metal thread, and wherein the support structure is preferably embedded in the first material or arranged on the inner side of the tube.

A fourteenth embodiment relates to a device according to one of the preceding embodiments, wherein the first medically acceptable material comprises a polymer, wherein the polymer preferably comprises polyether urethane or ethylene propylene diene rubber.

A fifteenth embodiment relates to a device according to one of the preceding embodiments, further comprising a fluid connector which is arranged, preferably detachably arranged, at the first end of the tube.

A sixteenth embodiment relates to an embodiment according to the fifteenth embodiment, wherein the fluid connector comprises a Luer-Lock connector and/or a check valve.

A seventeenth embodiment relates to a device according to one of the preceding embodiments, wherein the second end is closed or closable fluid-tightly, preferably by welding, fusing or by means of a plug or screw cap.

An eighteenth embodiment relates to a device according to one of the preceding embodiments, wherein the slits can be produced or are produced by severing portions of the tube without removing material from the tube, for example by punching with a blade.

A nineteenth embodiment relates to a device according to one of the preceding embodiments, wherein the device is designed and configured to permanently change the length of the tube, for example by removing a tube portion at the second end of the tube.

A twentieth embodiment relates to a device according to one of the preceding embodiments, wherein the tube is formed in one piece, and wherein the device does not comprise a second tube.

A first embodiment of a further aspect relates to a kit comprising a device according to one of the preceding embodiments and a means for introducing a medical fluid which can be connected to the first end of the tube.

A second embodiment of the kit according to the preceding embodiment further comprises a means for closing the second end of the tube.

A third embodiment relates to a kit according to the first or second embodiment, further comprising a medical fluid which comprises an active ingredient, wherein the active ingredient is preferably selected from the group consisting of an antibiotic, an antimycotic, an antitumor active ingredient, an osteoinductive active ingredient, and an anti-inflammatory active ingredient.

A fourth embodiment relates to a kit according to one of the preceding embodiments, further comprising a trocar, wherein the trocar is connectable, preferably detachably connectable, to the first end of the tube.

Another aspect relates to a medical therapy method, wherein the therapy method comprises administering a medical fluid to a patient using a device of the preceding embodiments or a kit of one of the preceding embodiments.

Another aspect relates to a medical active ingredient for use in bone surgery, wherein the active ingredient is selected from the group consisting of an antibiotic, an antimycotic, an antitumor active ingredient, an osteoinductive active ingredient, and an anti-inflammatory active ingredient, wherein the method comprises contacting a device of the preceding embodiments or a kit of one of the preceding embodiments with a surgical wound of a patient and locally administering the active ingredient as a medical fluid to the surgical wound using the device or kit.

### BRIEF DESCRIPTION OF THE DRAWINGS

FIG. 1 shows a cross-sectional view of a detail of the tube of a device according to the invention;

FIG. 2 shows an embodiment of a device according to the invention;

FIG. 3 shows a fluid connector that can be detachably connected to a tube of the device;

FIG. 4 shows a fluid connector with a check valve;

FIG. 5 shows the second end of a tube of a device according to the invention, which can be closed with a plug;

FIG. 6 shows the second end of a tube of a device according to the invention, which comprises a support structure in the lumen of the tube;

FIG. 7 shows the second end of a tube of a device according to the invention, wherein the second end comprises a fused and thereby closed tube wall;

FIG. 8 shows the second end of a tube with a second material embedded in the tube wall;

FIG. 9 shows a cross-sectional view of the second end of a tube with a second material embedded in the tube wall, wherein the second material is arranged in strand form in the tube;

FIG. 10 shows a cross-sectional view of the second end of a tube with a second material embedded in the tube wall, wherein the second material is arranged in a coaxially encircling manner in the tube;

FIG. 11 shows a longitudinal sectional view of a detail of a tube, wherein two slits are arranged offset from each other;

FIG. 12 shows a cross-sectional view of a detail of a tube, wherein two slits are arranged offset from each other;

FIG. 13 shows a longitudinal sectional view of a detail of a tube, wherein the slits in the tube are closed;

FIG. 14 shows a longitudinal sectional view of a detail of a tube, wherein the slits in the tube are open; and,

FIG. 15 shows a kit comprising a device described herein, a means for introducing a medical fluid and a means for closing the second end of the tube.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

“Operatively connected” or “operatively connectable” means herein that two elements appertaining thereto have a functional relationship to one another. For example, a first element may be configured to control or move a second element through such an operative connection. The term “control” here also comprises blocking or enabling a function, for example allowing or restricting the movement or other function of an element.

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, all the features described herein in connection with a method are in principle also considered to be applicable to the products, devices, kits and uses described herein, and vice versa. All such considered combinations are not explicitly listed in all instances, simply in order to keep the description brief. Technical solutions known to be equivalent to the features described herein are also intended in principle to be encompassed by the scope of the invention.

The technical norms and standards described herein, for example in conjunction with test procedures, refer to the version current on the priority date of the present application.

One embodiment of the invention relates to a device for dispensing a medical fluid to a patient, comprising a tube, wherein the tube comprises a first end for receiving a fluid into the tube; wherein the tube comprises a second end configured to retain a fluid in the tube; one or more slits for dispensing a fluid from the tube, wherein the slits each form a passage extending from an inner side of the tube to an outer side of the tube; and wherein the slits are configured to reversibly open depending on the pressure of a fluid within the tube so that the fluid is dispensed from the slits.

The device is preferably designed and configured to dispense a medical fluid to a patient. The term “fluid” herein includes aqueous and non-aqueous liquids, gases, and mixtures thereof. A “medical fluid” refers herein to a fluid which is intended for medical use and has a medicinal effect.

The term “medical fluid” herein refers in particular to aqueous and non-aqueous liquids which may contain dissolved active ingredients, in particular pharmaceutical active ingredients, or which may themselves have a medicinal effect. This term also includes gases and gas-liquid mixtures that can exert a pharmacological effect in the human or animal organism. In one embodiment, the medical fluid comprises an active ingredient. In one embodiment, the active ingredient is selected from the group consisting of an antibiotic, an antimycotic, an antitumor active ingredient, an osteoinductive active ingredient, and an anti-inflammatory active ingredient. In one embodiment, the active ingredient is an antibiotic. In one embodiment, the antibiotic is selected from the group consisting of penicillins, cephalosporins, carbapenems, quinolones, macrolides, lincosamides, aminoglycosides and glycopeptides. Examples of penicillins are amoxicillin and benzylpenicillin. Examples of cephalosporins are ceftriaxone and cefuroxime. Examples of carbapenems are meropenem and imipenem. Examples of quinolones are ciprofloxacin and levofloxacin. Examples of macrolides are azithromycin and clarithromycin. Examples of glycopeptides are vancomycin and teicoplanin. Examples of aminoglycosides are gentamicin and tobramycin. An example of an aminoglycoside is clindamycin.

In one embodiment, the active ingredient is an antimycotic. Examples of antimycotics comprise polyenes (e.g., amphotericin B, nystatin, natamycin), azoles (e.g., fluconazole, voriconazole), echinocandins (e.g., caspofungin, micafungin), and allylamines (e.g., terbinafine). In one embodiment, the active ingredient is an antitumor active ingredient (cytostatic). Examples of antitumor active ingredients (cytostatics) comprise alkylating substances, antimetabolites, natural products, protein kinase inhibitors and monoclonal antibodies.

Examples of alkylating substances comprise cyclophosphamide, melphalan and busulfan. Examples of antimetabolites comprise methotrexate, 5-fluorouracil, and gemcitabine. Examples of natural products comprise paclitaxel, doxorubicin and vincristine. Examples of protein kinase inhibitors comprise imatinib, gefitinib, and sunitinib. Examples of monoclonal antibodies comprise rituximab, trastuzumab and bevacizumab.

In a further embodiment, the active ingredient is an osteoinductive active ingredient. Examples of osteoinductive active ingredients comprise bone morphogenetic proteins (BMPs), parathyroid hormone-related peptides, anti-sclerostin antibodies, and growth factors. Examples of bone morphogenetic proteins comprise BMP-2 and BMP-7. An example of a parathyroid hormone-related peptide is teriparatide (PTH 1-34). An example of an anti-sclerostin antibody is romosozumab. Examples of growth factors comprise fibroblast growth factors (FGFs) and platelet derived growth factor (PDGF).

In a further embodiment, the active ingredient is an anti-inflammatory active ingredient. Examples of anti-inflammatory active ingredients comprise nonsteroidal anti-inflammatory drugs (NSAIDs), glucocorticoids, selective COX-2 inhibitors, biologics (e.g., TNF-α inhibitors), and Janus kinase inhibitors. Examples of nonsteroidal anti-inflammatory drugs (NSAIDs) comprise ibuprofen, diclofenac, and naproxen. Examples of glucocorticoids comprise prednisone, dexamethasone, and hydrocortisone. Examples of selective COX-2 inhibitors comprise celecoxib and etoricoxib. Examples of TNF-α inhibitors comprise infliximab, adalimumab, and etanercept. Examples of Janus kinase inhibitors comprise tofacitinib and baricitinib.

In one embodiment, the active ingredient is suitable for treating a bone disease. In one embodiment, the active ingredient is selected from the group of bisphosphonates (e.g., alendronate, zoledronate), calcitonin, selective estrogen receptor modulators (e.g., raloxifene) and strontium ranelate. In one embodiment, the active ingredient comprises hyaluronic acid or a corticosteroid (e.g., betamethasone, triamcinolone). In one embodiment, the active ingredient comprises a calcium salt. Examples of suitable calcium salts comprise calcium phosphates and calcium sulfates. Examples of calcium phosphates comprise beta-TCP and hydroxyapatite.

The device comprises a tube that can be filled with a medical fluid. The tube is preferably designed and configured to receive a medical fluid in an interior of the tube. The hollow interior of the tube is also called the “lumen.”

The tube comprises a medically acceptable material. “Medically acceptable” refers here to the property of a material not to have harmful effects on the human body or other biological systems. This means that a material can be introduced into the body safely and without unwanted side effects, without endangering the patient's health.

The tube comprises a first end for receiving a fluid into the tube.

One embodiment relates to a device for dispensing a medical fluid to a patient, comprising a tube comprising a first medically acceptable material with a Shore A hardness in the range of 75 to 95, wherein the tube has an inner diameter (ID) and an outer diameter (AD), wherein the ratio of [ID:AD] is in the range of [1:1.8] to [1:2.5]; wherein the tube comprises a first end for receiving a fluid into the tube; wherein the tube comprises a second end configured to retain a fluid in the tube; one or more slits for dispensing a fluid from the tube, wherein the slits each form a passage extending from an inner side of the tube to an outer side of the tube; and wherein the slits are configured to reversibly open depending on the pressure of a fluid within the tube so that the fluid is dispensed from the slits.

If the ratio of the inner diameter to the outer diameter of the tube [ID:AD] is in the range of [1:1.8] to [1:2.5], in particular for a tube made of a material with a Shore A hardness in the range of 75 to 95, this can reduce or prevent undesirable stretching of the tube when the interior of the tube is filled with a fluid of which the pressure is significantly higher than the ambient pressure.

This eliminates the need for an additional stabilizing tube layer.

If the ratio of the inner diameter to the outer diameter of the tube [ID:AD] is in the range of [1:1.8] to [1:2.5], it is also possible to ensure that no excessive force is required when filling the tube with a fluid and pressurizing the fluid.

The tube comprises a first material. The first material has a Shore A hardness in the range of 75 to 95. In one embodiment, the first material has a Shore A hardness of 80 to 90, for example about 85. The Shore A hardness is determined according to ASTM D2240.

In one embodiment, the tube comprises a mass fraction of the first material of at least 50%, preferably at least 60%, 70%, 80% or at least 90%. This mass fraction is calculated without taking into account any elements detachably connected to the tube, such as a fluid connector or plug, or the contents of the tube interior, such as a medical fluid. In one embodiment, the tube consists substantially entirely of the first material. The first material is preferably a polymer, in particular an elastomer. Preferably, the first material comprises or consists of a medically acceptable elastomer. Examples of medically acceptable elastomers comprise silicone elastomers, thermoplastic elastomers (TPEs), polyisoprene, butyl rubber, nitrile rubber, ethylene propylene diene monomer (EPDM), chloroprene rubber, fluoroelastomers, perfluoroelastomers, and polyacrylate elastomers. Examples of silicone elastomers comprise polydimethylsiloxane (PDMS) and liquid silicone rubber (LSR).

Examples of thermoplastic elastomers (TPEs) comprise styrene block copolymers (SBCs) such as styrene-ethylene-butylene-styrene (SEBS), thermoplastic polyurethanes (TPU), and thermoplastic copolyesters (TCE). Examples of polyisoprene comprise natural rubber and synthetic polyisoprene. Examples of butyl rubber comprise bromobutyl rubber (BIIR) and chlorobutyl rubber (CIIR). A preferred polyurethane is a polyether urethane. Polyether urethanes can be produced by polyaddition reaction of a polyetherpolyol with a diisocyanate.

In one embodiment, the first material has a thermoplastic elastomer. In one embodiment, the first material consists of a thermoplastic elastomer. In one embodiment, the first material has a polyether urethane or ethylene propylene diene rubber. In one embodiment, the first material comprises a polyether urethane. In one embodiment, the first material consists of polyether urethane.

In one embodiment, the first material has only a single elastomer, i.e., no second elastomer is mixed with the first material. In one embodiment, the first material has at least two different materials, for example two different elastomers. This allows, for example, the hardness of the first material to be set to a desired target value. The first material may comprise a copolymer. The copolymer may be a thermoplastic elastomer. Preferred are copolymers which comprise a soft segment and a hard segment in their molecular chain. The ratio between the soft segment and the hard segment within the molecular chain of the copolymer can be used to adjust the physical properties of such a copolymer, for example the Shore A hardness of the copolymer. The hard segment can be connected to the soft segment using a connector (linker).

Furthermore, the first material can comprise an additive to adjust the hardness. Examples of such additives are fillers and plasticizers. Examples of fillers comprise silica, titanium dioxide, calcium carbonate, barium sulfate and carbon.

Examples of plasticizers comprise adipates, trimellitates, citrate-based plasticizers, and esters of polyhydric alcohols.

For example, TOTM (tris(2-ethylhexyl)trimellitate), DINCH (diisononylcyclohexane-1,2-dicarboxylate), ATBC (acetyltributylcitrate), or DEHA (di(2-ethylhexyl)adipate) can be used as plasticizers.

In one embodiment, the first material is free of plasticizers. In one embodiment, the first material is free of fillers. In one embodiment, the first material is free of endocrine disrupting substances such as phthalates or bisphenols.

In one embodiment, the first material may further comprise a lubricant. Preferably, the lubricant is medically acceptable. Preferably, the lubricant is free of polyhalogenated substances and silicones. In one embodiment, the lubricant has a natural product, for example a lipid, a triglyceride or a biopolymer.

The first material may have a Young's modulus of elasticity of 1.2×10\hat{ }7 Pa to 2.1 x×10\hat{ }7 Pa, for example 1.3×10\hat{ }7 Pa to 2.0 x×10\hat{ }7 Pa, 1.4×10\hat{ }7 Pa to 1.9 x×10\hat{ }7 Pa, 1.5×10\hat{ }7 Pa to 1.8 x×10\hat{ }7 Pa, or 1.5×10\hat{ }7 Pa to 1.7 x×10\hat{ }7 Pa. In one embodiment, the first material may have a Young's modulus of elasticity of about 1.6×10\hat{ }7 Pa. In one embodiment, the first material may have a Young's modulus of elasticity of 2000 to 2500 psi. The latter corresponds approximately to 1.4×10\hat{ }7 Pa to 1.7×10\hat{ }7 Pa.

The Young's modulus of elasticity can be determined according to ASTM D412.

The first material is preferably sterilizable using common sterilization processes, i.e., resistant to UV radiation, gamma radiation and treatment with ethylene oxide within the scope of these processes.

The first material is preferably moldable using standard extrusion and/or injection molding processes.

The tube has an inner diameter (ID) and an outer diameter (AD).

Herein, the term “inner diameter” refers to the measurement of the straight-line distance between two opposite inner surfaces of the tube. This measurement is determined along the central longitudinal axis of the tube. The inner diameter is determined when the tube is relaxed, i.e., when no outer forces such as tension, pressure or torsion act on the tube that could change its original shape. The measurement only takes into account the clear width of the tube.

Similarly, the term “outer diameter” refers to the measurement of the straight-line distance between two opposite outer surfaces of the tube. This measurement is determined perpendicular to the central longitudinal axis of the tube. The outer diameter is measured when the tube is in a relaxed state.

Preferably, the ratio of inner diameter to outer diameter has a value in the range of [1:1.8] to [1:2.5], i.e., the outer diameter has a value which is 1.8 times to 2.5 times the inner diameter. In some embodiments, the ratio of inner diameter to outer diameter has a value in the range of [1:1.8] to [1:2.5]; [1:1.9] to [1:2.4]; [1:2.0] to [1:2.3]; or [1:2.1] to [1:2.2].

The tube comprises a first end for receiving a fluid into the tube. This means that a fluid can be introduced into the tube at the first end of the tube. For this purpose, the first end of the tube can comprise a fluid connector. The fluid connector can be used to connect a vessel or a fluid-conducting connection to receive a liquid into the tube. An example of a fluid connector is a Luer-Lock connector. Using such a connector, a commercially available Luer-Lock syringe can be connected to the first end of the tube in a liquid-tight and fluid-conducting manner. The fluid connector can be detachably connectable to the tube. The fluid connector can be operatively connectable to the tube. For this purpose, the fluid connector can comprise a substantially cylindrical nozzle with a thickening in order to form a frictionally engaged connection with the tube.

In one embodiment, the fluid connector may comprise a check valve. The check valve is preferably designed to prevent fluid from escaping from the tube through the fluid connector. Thus, a fluid can be introduced into the tube through the fluid connector without the fluid flowing back through the fluid connector.

The tube comprises a second end. The second end is designed to retain a fluid in the tube. This means that the lumen of the tube in the region of the second end is fluid-tight or can be closed fluid-tightly. For example, the tube can be closed at the second end by a plug or by a fused or glued tube wall.

The presence of slits in the tube wall remains unaffected, i.e., slits can also be provided in the region of the second end, as described herein.

This ensures that a fluid can only exit the tube through the slits in the tube wall and that the dispensation of a fluid can be controlled depending on the pressure.

The tube comprises one or more slits for dispensing a fluid from the tube. The slits each form a passage that extends from an inner side of the tube to an outer side of the tube. This means that the slits represent a breakthrough in the tube wall. The slits are configured to reversibly open depending on the pressure of a fluid within the tube so that the fluid is dispensed from the slits. This allows the slits to function as valves that can open and close depending on the pressure. The slits are preferably designed such that they comprise slit walls that touch each other when closed and preferably comprise a common contact surface over the entire region of the slit walls when closed.

This prevents the slits from becoming blocked, as they are only open when fluid is flowing out of the tube through the slits simultaneously. Ingrowth of cells and penetration of tissue components can also be prevented in this way. Due to the advantageous design of the device according to the invention, additional coatings, complex geometric designs and/or the addition of lubricants or anticoagulants (e.g., heparin), which may be necessary in designs not according to the invention, can therefore be dispensed with. Furthermore, flushing of the device in the implanted state may be dispensed with.

By a suitable choice of the tube material and/or the geometric design described herein, in particular with regard to the wall thickness of the tube and the length, depth and/or arrangement of the slits, the pressure-dependent opening characteristics of the slits can be achieved or improved on the one hand while simultaneously ensuring the stability of the device on the other. In particular, irreversible deformation or tearing of the tube in the region of the slits can be effectively prevented. Furthermore, the embodiments described herein can achieve a dispensing quantity of the fluid that is easy for the medical user to handle. In addition, a design of the tube according to the invention can allow for an opening characteristic of the slits that allows the slits to be opened at a limit pressure that can be easily built up manually using a commercially available plastics syringe. This is the case, for example, at an overpressure of approximately 1 bar.

In one embodiment, the slits are arranged within the first material. In one embodiment, the slits are arranged in the tube such that their opening characteristics with respect to the limit pressure at which the slits open result from the material properties of the first material.

In one embodiment, the tube comprises a first material and a second material, wherein the opening characteristic of the slits is independent of the second material.

In one embodiment, the tube comprises a first tube region at the first end of the tube, which comprises a homogeneously closed wall without slits.

In one embodiment, the tube further comprises a second tube region at the second end of the tube comprising a plurality of slits. In one embodiment, the tube comprises no slits in the first tube region and comprises a plurality of slits in the second tube region.

In one embodiment, the device according to the invention is designed and configured to dispense a medical fluid exclusively via the slits. Preferably, the device is designed and configured to dispense the fluid via a plurality of slits at different positions along the longitudinal axis of the tube. In one embodiment, the device is designed and configured to dispense a medical fluid exclusively in a pressure-dependent manner via the slits. Further preferably, the device is designed and configured to dispense the same volume flow of a fluid through a plurality of slits simultaneously. The term “volume flow” refers to the volume of a fluid per unit of time that is dispensed from the tube to the outside through a corresponding slit.

In one embodiment, the slits are designed and configured to open above a limit pressure of a fluid inside the tube, which is 1 bar (10\hat{ }5 Pa) higher than the pressure outside the tube, and to close fluid-tightly below this limit pressure. This means that the slits are closed when the overpressure of a fluid in the tube is less than 1 bar and the slits are open when the overpressure of a fluid in the tube is 1 bar or higher. “Overpressure” refers here to the difference between the atmospheric pressure and the pressure of the fluid in the tube.

In one embodiment, this limit pressure is about 1 bar, about 1.2 bar, about 1.3 bar, about 1.4 bar, or more than 1.4 bar.

In one embodiment, the slits are configured to reversibly open and close by an elastic restoring force of the first material of the tube. The slits can be opened by an overpressure of a fluid in the tube, as the material of the tube, in particular the first material of the tube, is pushed apart by the fluid.

The stability of the slits and the limit pressure at which the slits open may depend on the dimensions of the tube and the slits.

The slits have a slit length L. In one embodiment, the ratio of the slit length L to the inner diameter ID of the tube [L:ID] is in the range of [1:2.2] to [1:2.9]. This means that the inner diameter of the tube is 2.2 to 2.9 times the slit length. The ratio of the slit length L to the inner diameter ID of the tube [L:ID] can, for example, be in the range from [1:2.3] to [1:2.8], from [1:2.4] to [1:2.7], or from [1:2.5] to [1:2.6]. In one embodiment, the ratio of the slit length L to the inner diameter ID of the tube [L:ID] is approximately [1:2.5].

In one embodiment, the ratio of the slit length L to the outer diameter AD of the tube [L:AD] is in the range of [1:3.5] to [1:5.5]. This means that the outer diameter of the tube is 3.5 to 5.5 times the slit length. For example, the ratio of the slit length L to the outer diameter AD of the tube [L:AD] is in the range of [1:3.6] to [1:5.4], [1:3.7] to [1:5.3], [1:3.8] to [1:5.2], [1:3.9] to [1:5.1], [1:4.0] to [1:5.0], [1:4.1] to [1:4.9], [1:4.2] to [1:4.8], [1:4.3] to [1:4.7], or [1:4.4] to [1:4.6]. In one embodiment, the ratio of the slit length L to the outer diameter AD of the tube is approximately 4.5.

In one embodiment, the device is designed and configured to change the outer diameter of the tube AD by less than 10% when the pressure of a fluid inside the tube increases from 0 to 1 bar (10\hat{ }5 Pa) higher than the pressure outside the tube. This can prevent the tube from expanding when a fluid in the tube reaches the limit pressure at which the slits open. The dimensional stability of the tube even at increased fluid pressure can prevent irritation of sensitive tissue.

In some embodiments, the outer diameter of the tube changes accordingly by less than 9%, 8%, 7%, 6% or 5% when the overpressure of the fluid increases from 0 to 1 bar.

In one embodiment, the device is designed and configured to change the length of the tube by less than 10%, preferably less than 9%, 8%, 7%, 6% or 5%, when the pressure of a fluid inside the tube increases from 0 to 1 bar (10\hat{ }5 Pa) higher than the pressure outside the tube.

In one embodiment, the tube comprises a plurality of slits, each arranged at a distance A from one another on the outer side of the tube, and each having a slit length L along the outer side of the tube, wherein the ratio of distance (A) to slit length (L) [A:L] is at least [10:1]. This means that the shortest distance between two slits is at least ten times the slit length.

The distance between two slits is determined starting from the adjacent ends of the slits. The distance measurement between two adjacent slits is determined using a measurement methodology that does not determine the centric distance between the centers of the slits, but the minimum distance between the outermost boundaries of the slits. This determination implies a measurement that focuses on the shortest physically existing distance between the closely spaced interfaces of the slits, independent of the shape, orientation and position of the centers of the slits relative to each other. Accordingly, the minimum distance is determined exactly at the position where the closest points of two adjacent slits have the smallest possible spatial distance from each other. In some embodiments, the ratio of distance (A) to slit length (L) [A:L] is at least [15:1], [20:1], or [30:1].

This can prevent the slits from tearing open in the longitudinal direction and excessive radial expansion of the tube.

In one embodiment, the tube comprises a second material which has a higher Shore A hardness than the first material. For example, the Shore A hardness of the second material may have a value which is at least 1.1 times, 1.2 times, 1.3 times, 1.4 times, 1.5 times or 2 times the Shore A hardness of the first material. The second material may comprise an elastomer. An elastomer is a plastics material that has rubber-elastic properties. The Shore A hardness of the second material can be adjusted by means of plasticizers and/or fillers, as described above in conjunction with the first material. Accordingly, it is possible that the first material and the second material differ only in the content of such plasticizers and/or fillers. The first material and the second material can therefore comprise the same or different elastomers. In one embodiment, the first material comprises a first elastomer and the second material comprises a second elastomer. In one embodiment, the second material does not comprise any elastomer contained in the first material. In one embodiment, the first material and the second material comprise the same elastomer.

The second material may comprise a thermoplastic elastomer. The second material may comprise a copolymer. The copolymer may be a thermoplastic elastomer. Preferred are copolymers which comprise a soft segment and a hard segment in their molecular chain. The ratio between the soft segment and the hard segment within the molecular chain of the copolymer can be used to adjust the physical properties of such a copolymer. The hard segment can be connected to the soft segment using a connector (linker).

Furthermore, the first material can comprise an additive to adjust the hardness, as described herein.

In one embodiment, the second material is free of plasticizers. In one embodiment, the second material is free of fillers. In one embodiment, the second material is free of endocrine disrupting substances such as phthalates or bisphenols.

In this embodiment, both the first material and the second material are free of plasticizers, fillers and endocrine disrupting substances. Other groups of products described herein, such as radiopaque agents, polymers or dyes, are not to be understood as “fillers.” Fillers are understood herein only to mean those substances of which the primary function is to adjust the hardness of a first material or a second material and which do not belong to one of the other groups of products described herein.

In one embodiment, the second material may further comprise a lubricant. Preferably, the lubricant is medically acceptable. Preferably, the lubricant is free of polyhalogenated substances and silicones.

In one embodiment, the first material comprises a polyether urethane and the second material comprises a polyether block amide.

The second material can be arranged as a coaxially encircling layer in the tube or on a surface of the tube. The second material can be arranged as a strip parallel to a longitudinal axis of the tube. The second material can be located on the inner side of the tube or on the outer side of the tube. The second material may be embedded in the first material, for example completely embedded therein. In one embodiment, the second material is arranged in the tube in such a way that it does not influence the opening and closing characteristics of the slits, in particular with respect to the limit pressure of a fluid at which the slits open. The second material can be designed to stabilize the overall structure of the tube, in particular to stabilize the longitudinal or radial expansion of the tube when there is a change in pressure of a fluid received in the tube.

In one embodiment, the second material comprises a radiopaque agent. Examples of suitable radiopaque agents comprise barium sulfate and tungsten. In one embodiment, the second material comprises a dye. The dye can have an absolute emission maximum in the range of 490 nm to 575 nm. The dye can improve the visibility of the tube, especially when the tube is in the region of a surgical wound. Dyes with a significant light emission in the range of 490 nm to 575 nm can provide a particularly good contrast to reddish colored body tissue for the human eye.

In one embodiment, the second material comprises both an elastomer and a radiopaque agent. The radiopaque agent can be embedded in the elastomer.

In one embodiment, the slits are arranged parallel to a longitudinal axis of the tube. This can achieve improved stability of the slits and the tube. In one embodiment, at least 40%, at least 50% or at least 90% of all slits are arranged parallel to a longitudinal axis of the tube.

The slits may be arranged circumferentially pointing in different directions to dispense fluid from the tube in different directions. This can allow for a more spatially uniform dispensation of a fluid. For example, in one embodiment, in a first longitudinal position in the direction of the longitudinal axis of the tube, a first slit is arranged at a first radial position, and in a second longitudinal position in the direction of the longitudinal axis of the tube, a second slit is arranged at a second radial position. In one embodiment, the first radial position forms an angle of approximately 90° to the second radial position. This corresponds to an arrangement in which, in a cross-sectional view of the tube, the first slit is arranged, for example, in the “12 o'clock position” and the second slit is arranged in the “9 o'clock position” or “3 o'clock position.” In one embodiment, the above statements refer to a first slit and a second slit in mutually adjacent longitudinal positions.

In one embodiment, the tube comprises pairs of slits, each located at the same longitudinal position in the direction of the longitudinal axis of the tube, but forming an angle of 180° to each other in their radial position. This corresponds to an arrangement in which, in a cross-sectional view of the tube, the first slit of this paired arrangement is arranged, for example, in the “12 o'clock position” and the second slit of this paired arrangement is arranged in the “6 o'clock position.”

In one embodiment, the two above-mentioned arrangements are implemented simultaneously, i.e., a first pair of slits forms, relative to a second pair of slits, an angle of approximately 90° with respect to the radial position, while within the first pair of slits and within the second pair of slits the two slits each form an angle of 180° with respect to one another.

In one embodiment, the tube comprises a support structure made of metal. In one embodiment, the support structure comprises a metal coating, a metal foil, a metal spiral or a metal thread. In one embodiment, the support structure is embedded in the first material. In one embodiment, the support structure is arranged on the inner side of the tube. Comparable support structures may alternatively or additionally be formed from the second material described herein.

In order to achieve a uniform dispensation of fluid from the tube, it is advantageous to close the second end of the tube. This means that the tube in particular does not comprise an opening at the second end which is larger than the maximum cross section of the opened slits.

Therefore, in one embodiment of the invention, the second end of the tube is sealed fluid-tightly. For this purpose, the tube can, for example, be welded or fused at the second end so that the interior of the tube comprises a fluid-conducting connection to the outside exclusively via the first end and the slits.

In one embodiment, the second end of the tube is closable. For example, the tube can be designed to be closed by a plug or screw cap at the second end. Such a closure may be included with the device.

In one embodiment, the tube is closed or closable at the second end in such a way that the second end is closed liquid-tightly even at a pressure of at least 1 bar, preferably at least 2 bar. The pressure-dependent opening of the slits remains unaffected.

According to the invention, the tube is designed to open the slits in a fluid-conducting manner or to close them fluid-tightly depending on the pressure of a fluid received therein. Such a function can be achieved cost-effectively by severing portions of the tube. Preferably, no material is removed from the tube, so that the tube can comprise a smooth and homogeneous surface in a configuration in which the slits are closed. Accordingly, one embodiment of the invention relates to a device described herein, in which the slits can be produced or are produced by severing portions of the tube without removing material from the tube. This can be done, for example, by cutting or punching with a blade. As a result, the slits can be formed as incisions within the wall of the tube. This can result in better lubrication, less damage to tissue, and the prevention of clogging or sticking of the openings by body tissue.

It may be advantageous to design the device so that the medical user can shorten the length of the tube before bringing the device into contact with a patient. In one embodiment, the device is therefore designed and configured to permanently change the length of the tube. This can be done, for example, by removing a tube portion at the second end of the tube, i.e., the tube can be cut off at the second end without affecting the function of the device.

Subsequently, as previously described, it may be advantageous to close the second end of the tube, for example with a plug.

As explained above, comparable devices are known in the prior art which necessarily comprise several tubes or several tube layers which can be moved relative to one another in order to ensure the stability of the device, to prevent expansion of the entire tube due to a pressurized fluid contained therein, and/or to adjust the region of fluid dispensation. The present invention, on the other hand, allows for a functional device even with a one-piece design of the tube, without the need for a further tube or a further, separate tube layer. A further embodiment therefore relates to a device described herein in which the tube is formed in one piece. In one embodiment, the device does not comprise a second tube. In one embodiment, the device comprises neither a second tube nor two mutually displaceable tube layers. In one embodiment, the first material and the second material together form a monolithic unit.

Another aspect of the invention relates to a kit comprising a device described herein and a means for introducing a medical fluid. The means for introducing a medical fluid is preferably connectable, preferably operatively connectable, to the first end of the tube. The means for introducing a medical fluid may, for example, comprise a syringe. Other standard devices can also be used to introduce a medical fluid into the tube. Other examples of means for introducing a medical fluid comprise fluid adapters, infusion pumps, and gravity infusion systems. Examples of fluid adapters are port systems and similar accesses, as well as spacers that allow the appropriate connection of a medicament container. Examples of infusion pumps comprise peristaltic pumps, syringe pumps, balloon pumps, piston pumps, and other pumps commonly used in the medical environment. The means for introducing a medical fluid is preferably designed to establish a fluid-tight connection with the first end of the tube. For example, the means for introducing a medical fluid and the first end of the tube can comprise matching elements of a Luer-Lock connection.

The kit may further comprise a means for closing the second end of the tube. For example, the kit may contain a plug with which the second end of the tube can be closed. In one embodiment, the kit contains a tool configured to close the second end of the tube. Such a tool can, for example, crimp, fuse, clamp or plug the tube to close it at the second end.

The kit may further comprise a medical fluid intended for administration by means of the device. The fluid preferably comprises an active ingredient. In one embodiment, the active ingredient is preferably selected from the group consisting of an antibiotic, an antimycotic, an antitumor active ingredient, an osteoinductive active ingredient, and an anti-inflammatory active ingredient. It may contain all of the active ingredients described herein as well as other active ingredients that can be administered in liquid form.

In a further embodiment, the kit further comprises a trocar. A trocar comprises a pointed end that allows it to penetrate tissue. Furthermore, a trocar comprises a shaft along which the trocar can be guided. The shaft may comprise a cylindrical cavity, similar to a cannula.

The trocar can preferably be connected to the first end of the tube. Preferably, the trocar can be detachably connected to the first end of the tube. Using the trocar, a medical user can insert the tube of the device into patient's tissue and thereby position and fix it at a desired location. A trocar can allow for gentle and precise penetration of a target tissue. In this case, a channel can be created to the desired administration site, allowing the device for administering a medical fluid to be positioned accordingly. The tube of the device can be inserted into the patient's tissue through the channel created with the help of the trocar. Preferably, the trocar is removable to allow a medical fluid to be received through the first end of the tube following positioning of the device using the trocar. For example, the trocar can be removed from the first end of the tube and replaced with a fluid connector, such as a Luer-Lock connector. A medical fluid can then be added to the tube using a Luer-Lock syringe, for example, and then dispensed to a target location on the patient's tissue using the device.

Another aspect of the invention relates to a medical therapy method, wherein the therapy method comprises administering a medical fluid to a patient using a device described herein or a kit described herein.

The therapy method may in particular comprise one, several or all of the steps described below:
if necessary, shortening the tube at the second end of the tube
if necessary, closing the second end of the tube
connecting a trocar to the first end of the device
penetrating a patient's tissue using the trocar to create access to a target location of the patient's tissue
inserting the tube through the access created with the trocar
bringing the tube into contact with a target location of the patient's tissue
removing the trocar from the first end of the tube
connecting a fluid connector to the first end of the tube
introducing a medical fluid into the tube, preferably via a fluid connector at the first end of the tube
pressurizing the medical fluid within the tube to open the slits and dispense the medical fluid through the slits to the patient's tissue.

In one embodiment, the therapy method particularly comprises pressurizing the medical fluid within the tube to open the slits and dispense the medical fluid to the patient's tissue through the slits. This means that the pressure of the medical fluid inside the tube is increased to a value at which the slits open due to the rubber-elastic properties of the first material.

Another aspect relates to a medical active ingredient for use in bone surgery, wherein the active ingredient is selected from the group consisting of an antibiotic, an antimycotic, an antitumor active ingredient, an osteoinductive active ingredient, and an anti-inflammatory active ingredient, wherein the method comprises contacting a device described herein or a kit described herein with a surgical wound of a patient and locally administering the active ingredient as a medical fluid to the surgical wound using the device or kit. Examples of such active ingredients are described herein.

For the purposes of this patent application, “bone surgery” means any surgical procedure involving the treatment of bone tissue of the human or animal body. This comprises, but is not limited to, interventions on the bone itself, the adjacent soft tissues, joints, and associated structures such as cartilage, tendons, ligaments, skeletal muscles and vessels. The term “bone surgery” includes, for example, traumatic surgery, corrective and constructive surgery, orthopedic surgery, implantation surgery, arthroscopic surgery, spine surgery and microsurgical procedures.

Traumatic surgery deals with the treatment of fractures of all kinds, dislocations, bony injuries, restoration of bone integrity and function after acute and chronic injuries.

Corrective and reconstructive surgery includes procedures to correct misalignments, deformities and defects in the bone, which may be of genetic, traumatic or disease-related origin. This also includes the reconstruction of bone defects after tumor resections and infection treatments.

Orthopedic surgery deals with the diagnosis, treatment, rehabilitation and prevention of diseases, disorders and injuries of the musculoskeletal system.

Implantation surgery comprises the insertion of orthopedic implants, prostheses, artificial joints, fixation elements such as screws, plates, nails, wires and anchoring systems to support or replace bone structures.

Arthroscopic surgery comprises minimally invasive procedures in joints to treat injuries or conditions such as arthritis, meniscus tears or cruciate ligament lesions.

Spine surgery comprises, for example, the correction of spinal deformities, the treatment of herniated discs, spinal fusions and the stabilization of spinal fractures.

Microsurgical procedures comprise procedures that require the use of a microscope for precise treatment of smaller bone structures, comprising nerve repair and vascular anastomosis.

In the context of the present invention, bone operations comprise, for example, surgical interventions in the region of joints, in particular hip joints, knee joints or shoulder joints.

Another aspect relates to a medical active ingredient for the therapy or prophylaxis of a bone disease, wherein the active ingredient is selected from the group consisting of an antibiotic, an antimycotic, an antitumor active ingredient, an osteoinductive active ingredient, and an anti-inflammatory active ingredient, wherein the method comprises the local administration of the active ingredient using a device or kit described herein as a medical fluid to a target tissue of a patient.

Bone disease may comprise infection, injury, degeneration, or inflammatory disease of a bone. Examples of bone diseases comprise rheumatism, arthritis, cancer, injuries to bone or cartilage, joint infections, and osteomyelitis.

### EXAMPLES

The invention is further illustrated below using examples which are, however, not to be understood as limiting. It will be apparent to a person skilled in the art that other equivalent means may be used similarly in place of the features described here.

The following tubes were produced by extrusion from commercially available medical polyether urethane with Shore A hardnesses of 75, 85 and 95:

| | | Inner | Outer | Ratio of inner | |
|---|---|---|---|---|---|
| Tube | Shore A | diameter | diameter | diameter to outer | |
| sample | hardness | [mm] | [mm] | diameter | |
| 1 | 75 | 2.43 | 3.65 | 1 | 1.5 |
| 2 | 75 | 2.03 | 3.65 | 1 | 1.8 |
| 3 | 75 | 1.83 | 3.65 | 1 | 2.0 |
| 4 | 75 | 1.46 | 3.65 | 1 | 2.5 |
| 5 | 75 | 1.21 | 3.65 | 1 | 3.0 |
| 6 | 85 | 2.43 | 3.65 | 1 | 1.5 |
| 7 | 85 | 2.03 | 3.65 | 1 | 1.8 |
| 8 | 85 | 1.83 | 3.65 | 1 | 2.0 |
| 9 | 85 | 1.46 | 3.65 | 1 | 2.5 |
| 10 | 85 | 1.21 | 3.65 | 1 | 3.0 |
| 11 | 95 | 2.43 | 3.65 | 1 | 1.5 |
| 12 | 95 | 2.03 | 3.65 | 1 | 1.8 |
| 13 | 95 | 1.83 | 3.65 | 1 | 2.0 |
| 14 | 95 | 1.46 | 3.65 | 1 | 2.5 |
| 15 | 95 | 1.21 | 3.65 | 1 | 3.0 |

Three trapezoidal ground blades with a blade length (length of the cutting edge parallel to the tube surface) of 0.5 mm, 0.7 mm and 1.0 mm were produced from tool steel. With these, the tube samples were slit at different distances at one point on the tube from two opposite sides. Preliminary tests with distilled water using a 5 ml syringe showed that a uniform release of liquid can be achieved with a blade length of 0.7 mm and 1.0 mm.

With a blade length of 0.5 mm, the amount of fluid released per slit was very small and the force required to operate the syringe was unacceptably high. Therefore, no further testing was carried out for the corresponding samples.

Furthermore, it was found that the hand force required to open the slits in the samples of examples 1, 6 and 11 was uncomfortably high when operating the syringe. Further tests were conducted on the remaining samples using a caliper to measure the expansion of the tube samples with a slit length of 0.7 mm and 1.0 mm. It was shown that at a pressure of 1.0 bar or more, the slits opened and the liquid escaped from the slits. During the liquid dispensation, the pressure was 1.4 bar to 2.0 bar, and this pressure was built up manually with a syringe. In further tests, it was observed that with a slit length of 0.7 mm and a distance between the slits of less than 7 mm, especially with a distance of 2 to 3 mm, temporary radial expansions and sometimes enlargements of the slits occurred when pressure was applied several times. Analogously, similar effects occurred with a slit length of 1.0 mm and a slit spacing of less than 10 mm.

The following tests were therefore carried out with a slit spacing of 7 mm for 0.7 mm slits and a slit spacing of 10 mm for 1.0 mm slits. The expansion from the opening of the slits was determined using a caliper. An expansion of more than 10% of the outer diameter was chosen as the limit value. Radial expansion less than 10% was rated as “+” and expansion greater than 10% was rated as “−”

| | | Slit | Ratio | | |
|---|---|---|---|---|---|
| Tube | | length | Slit | Inner | Outside |
| sample | Evaluation | [mm] | length | diameter | diameter |
| 2 | + | 0.7 | 1.0 | 2.9 | 5.2 |
| 3 | + | 0.7 | 1.0 | 2.6 | 5.2 |
| 4 | + | 0.7 | 1.0 | 2.1 | 5.2 |
| 5 | − | 0.7 | 1.0 | 1.7 | 5.2 |
| 2 | + | 1.0 | 1.0 | 2.0 | 3.6 |
| 3 | + | 1.0 | 1.0 | 1.8 | 3.6 |
| 4 | + | 1.0 | 1.0 | 1.5 | 3.6 |
| 5 | − | 1.0 | 1.0 | 1.2 | 3.6 |
| 7 | + | 0.7 | 1.0 | 2.9 | 5.2 |
| 8 | + | 0.7 | 1.0 | 2.6 | 5.2 |
| 9 | + | 0.7 | 1.0 | 2.1 | 5.2 |
| 10 | − | 0.7 | 1.0 | 1.7 | 5.2 |
| 7 | + | 1.0 | 1.0 | 2.0 | 3.6 |
| 8 | + | 1.0 | 1.0 | 1.8 | 3.6 |
| 9 | + | 1.0 | 1.0 | 1.5 | 3.6 |
| 10 | − | 1.0 | 1.0 | 1.2 | 3.6 |
| 12 | + | 0.7 | 1.0 | 2.9 | 5.2 |
| 13 | + | 0.7 | 1.0 | 2.6 | 5.2 |
| 14 | + | 0.7 | 1.0 | 2.1 | 5.2 |
| 15 | − | 0.7 | 1.0 | 1.7 | 5.2 |
| 12 | + | 1.0 | 1.0 | 2.0 | 3.6 |
| 13 | + | 1.0 | 1.0 | 1.8 | 3.6 |
| 14 | + | 1.0 | 1.0 | 1.5 | 3.6 |
| 15 | − | 1.0 | 1.0 | 1.2 | 3.6 |

### FIGURES

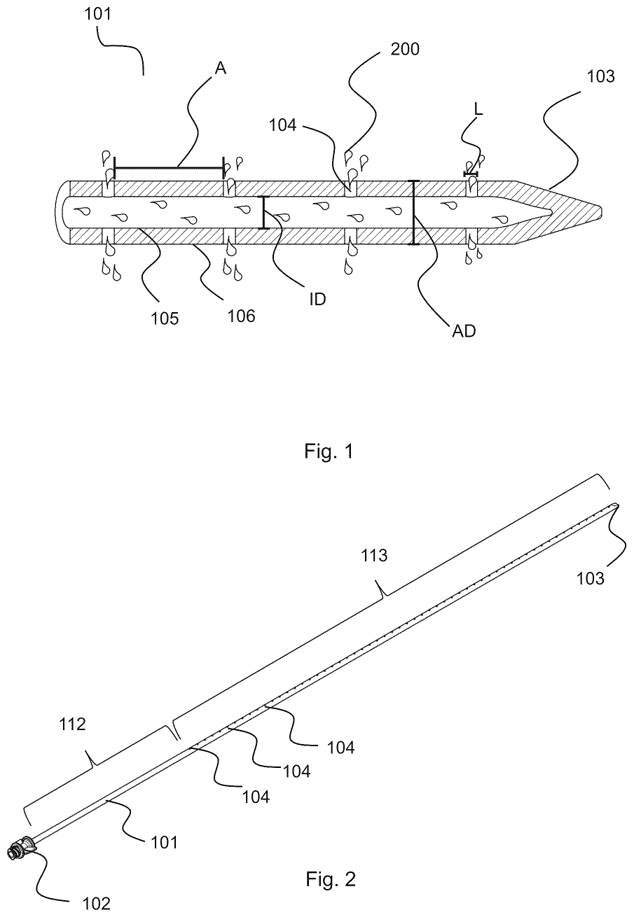

FIG. 1 shows a cross-sectional view of a detail of the tube 101 of a device 100 according to the invention. The tube 101 comprises a first end 102 (not shown in this figure) and a second end 103. In the wall of the tube 101 there are slits 104, each of which extends from an inner side 105 of the tube to an outer side 106 of the tube. Thus, the slits form 104 a fluid-conducting connection between the inner side 105 and the outer side 106 of the tube through which a fluid 200 from the interior of the tube 101 can be dispensed to the outside. The tube has an inner diameter ID which corresponds to the clear width of the tube. The tube still has an outer diameter AD which is a measure of the straight line distance between two opposite outer surfaces of the tube 101. The slits each have a slit length L. At the second end 103 the tube 101 is closed so that the fluid 200 can escape only through the slits 104.

FIG. 2 shows an embodiment of a device 100 according to the invention. The device comprises a first end 102 which is designed to receive a fluid into the tube 101. The tube comprises a first tube region 112 which comprises a homogeneously closed wall without slits. The tube also comprises a second tube region 113 which comprises a multitude of slits 104. The first end 102 borders on the first tube region 112 while the second end 103 borders on the second tube region 113.

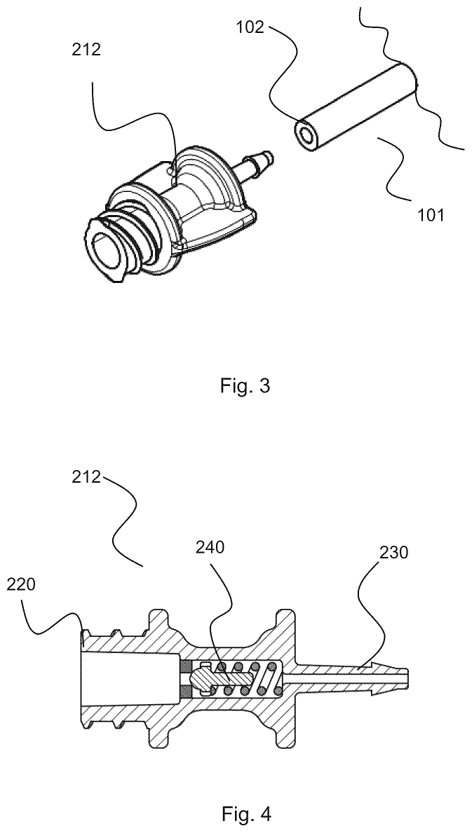

FIG. 3 shows a fluid connector 212, which can be detachably connected to a tube 101 of the device. The fluid connector is connectable to the first end 102 of the tube to allow a fluid to be received into the tube 101.

FIG. 4 shows a fluid connector 212 with a Luer-Lock connector 220, a tube connector 230, and a check valve 240. The Luer-Lock connector 220 for example allows for the device to be detachably connected to a syringe in order to introduce a medical fluid into the device. Simultaneously, the syringe can be used to pressurize the fluid inside the tube to open the slits. The tube connector 230 allows a detachable, fluid-conducting and liquid-tight connection with the first end 102 the tube 101. The tube connector 230 to this end can be inserted into the tube 101. The tube connector comprises a cylindrical nozzle with a thickening to form a frictionally engaged, liquid-tight connection with the tube.

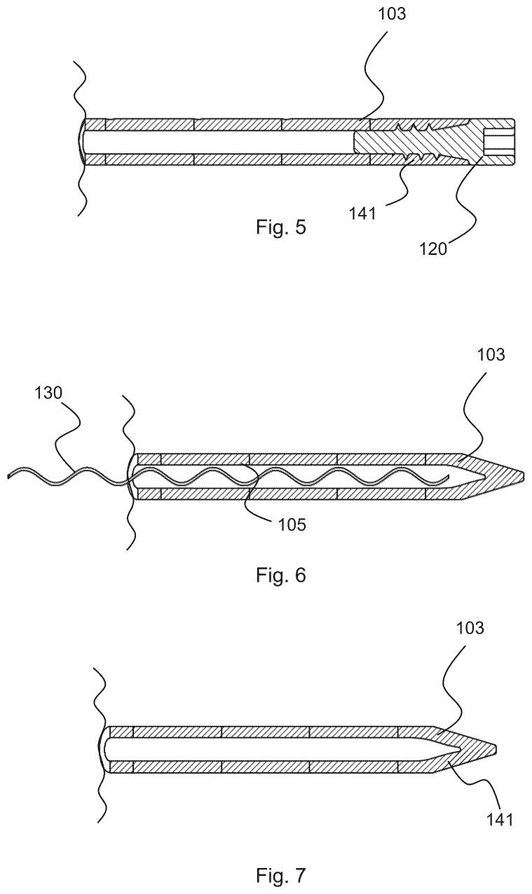

FIG. 5 shows the second end 103 of a tube of a device according to the invention, which can be closed with a plug 120. The plug 120 is provided here with a thread to create a frictionally engaged connection with the tube, so that the tube at the second end 103 can be sealed liquid-tightly. The plug can be similarly provided with circumferential elevations which can engage in the flexible first material 141 of the tube to seal the tube liquid-tightly.

FIG. 6 shows the second end 103 of a tube of a device according to the invention, which comprises a support structure 130 in the lumen of the tube. The support structure is designed here as a spiral-wound metal wire, which is arranged on the inner side 105 of the tube. The support structure 130 can increase the bending strength of the tube, so that, for example, the formation of kinks in the tube can be avoided.

FIG. 7 shows the second end of a tube 103 of a device according to the invention, wherein the second end 103 comprises a fused and therefore closed tube wall. In particular, the first material 141 can form a fused tube end.

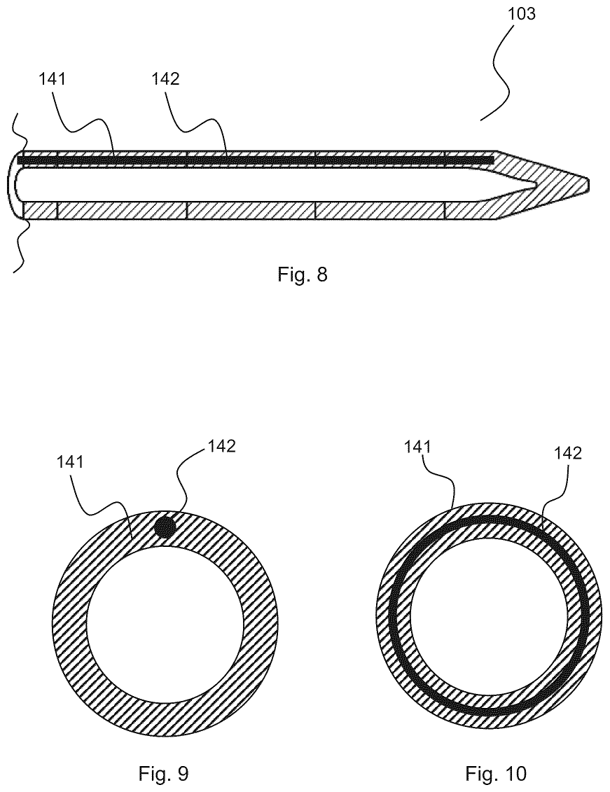

FIG. 8 shows the second end 103 of a tube with a second material 142 embedded in the tube wall. The second material 142 has a higher hardness than the surrounding first material 141 and extends in strand form parallel to the central longitudinal axis of the tube towards the second end 103 of the tube. This allows the geometry of the tube to be stabilized. In particular, expansion of the tube due to a pressurized liquid inside the tube can be reduced or avoided.

FIG. 9 shows a cross-sectional view of the second end of a tube 103 with a second material 142 embedded in the tube wall, wherein the second material 142 is arranged in strand form in the tube. The second material 142 is arranged here in strand form within the first material 141 and comprises a circular, closed cross section. The second material 142 is completely surrounded by the first material 141.

FIG. 10 shows a cross-sectional view of the second end of a tube 103 with a second material 142 embedded in the tube wall, wherein the second material 142 is arranged such that it runs in an encircling manner coaxially in the tube. The second material 142 here comprises a circular, open cross section. The second material 142 here forms a completely radially encircling layer, which is completely embedded in the first material 141. The first material 141 and the second material 142 together form a monolithic unit, so that the first material 141 and the second material 142 cannot be moved relative to each other.

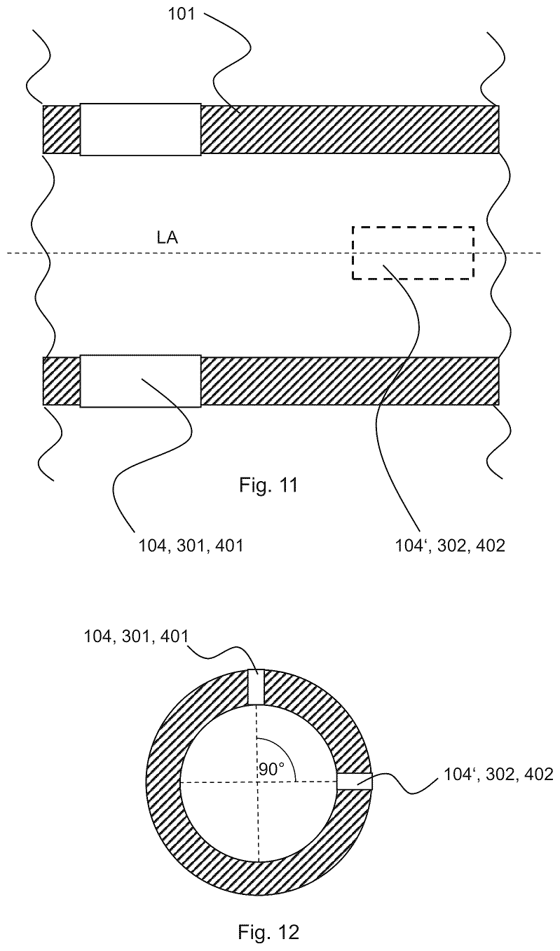

FIG. 11 shows a longitudinal sectional view of a detail of a tube 101, wherein two slits 104, 104′ are arranged offset from each other. A first slit 104 is at a first longitudinal position 301 and at a first radial position 401 of the tube. A second slit 104′ is at a second longitudinal position 302 and at a second radial position 402 of the tube. In this case, the first longitudinal position differs from the second longitudinal position, and the first radial position differs from the second radial position. The two slits 104, 104′ are offset from each other in the longitudinal direction of the tube, i.e., parallel to a longitudinal axis LA of the tube, and point in different radial directions, in order to dispense a medical fluid to spatially separated target regions.

FIG. 12 shows a cross-sectional view of a detail of a tube, wherein two slits 104, 104′ are arranged offset from each other. A first slit 104 is at a first longitudinal position 301 and at a first radial position 401 of the tube. A second slit 104′ is at a second longitudinal position 302 and at a second radial position 402 of the tube. Here, the first radial position 401 forms an angle of about 90° to the second radial position 402, wherein the legs of the angle intersect in the longitudinal axis of the tube.

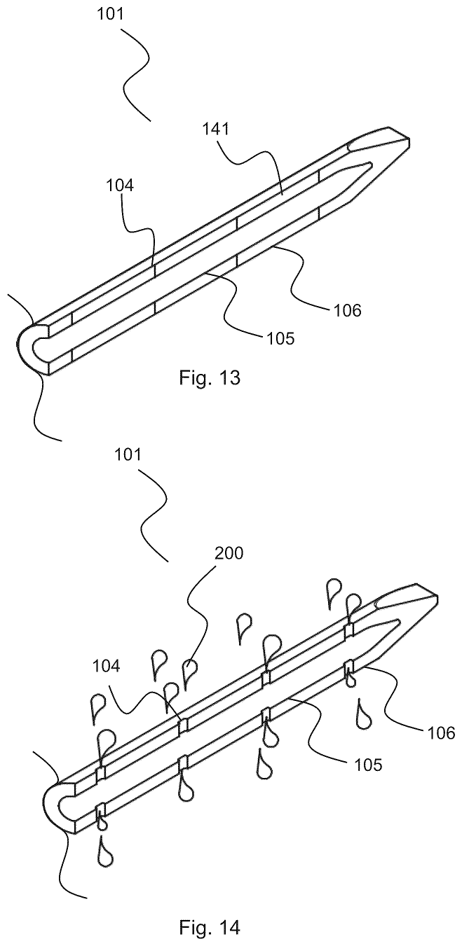

FIG. 13 shows a longitudinal sectional view of a detail of a tube 101, wherein the slits 104 in the tube are closed. The slits 104 extend from the inner side 105 of the tube to the outer side 106 of the tube. Due to the restoring force of the first material 141, the slits 104 are compressed and thus remain liquid-tight as long as no force acts on the tube.

FIG. 14 shows a longitudinal sectional view of a detail of a tube 101, wherein the slits 104 in the tube are open. A pressurized medical fluid 200, which is received in the tube, presses here against the inner side 105 of the tube so that the slits 104 are pushed apart and open. This allows the medical fluid 200 from the lumen of the tube to be dispensed to the outside through the slits 104. The opening state of the slits 104 can be controlled here by the pressure of the medical fluid.

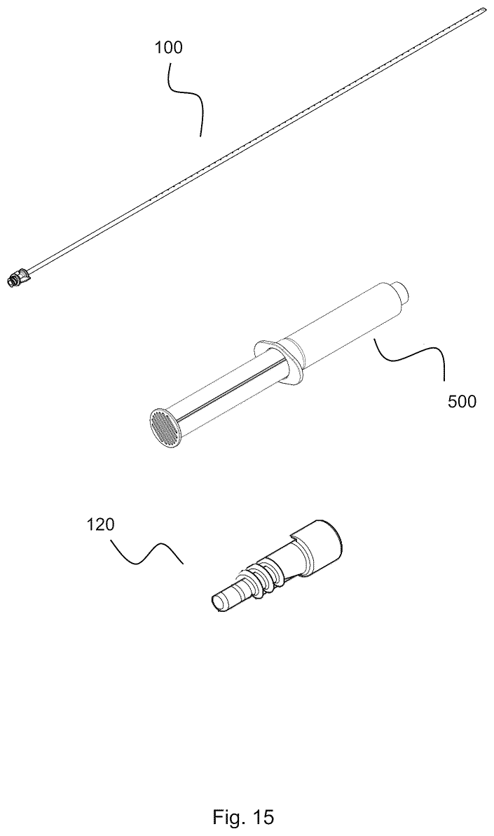

FIG. 15 shows a kit containing a device 100 described herein, a means 500 for introducing a medical fluid, which is designed here as a syringe, and a means 120 for closing the second end of the device, which is designed here as a plug. Using the syringe 500 a medical fluid can be introduced into the device 100, especially by connecting the syringe 500 with the first end of the tube of the device 100. The plug 120 can be used to seal the second end of the tube of the device 100 liquid-tightly. If necessary, a user can shorten the length of the tube before connecting the tube to the plug 120 at the second end of the tube of the device.

### LIST OF REFERENCE NUMERALS

100 Device
101 Tube
102 First end
103 Second end
104 Slit
105 Inner side of the tube
106 Outer side of the tube
112 First tube region
113 Second tube region
120 Means for closing the second end
130 Support structure
141 First material
142 Second material
200 Fluid
212 Fluid connector
220 Luer-Lock connector
230 Tube connector
240 Check valve
301 First longitudinal position
302 Second longitudinal position
401 First radial position
402 Second radial position
500 Means for introducing a medical fluid
A Distance between two slits on the outer side of the tube
AD Outer diameter of the tube
L Slit length
LA Longitudinal axis of the tube
ID Inner diameter of the tube

1. A device (100) for dispensing a medical fluid to a patient, comprising
a tube (101) comprising a first medically acceptable material (141) with a Shore A hardness in the range of 75 to 95,
wherein the tube (101) has an inner diameter (ID) and an outer diameter (AD),
wherein the ratio of [ID:AD] is in the range of [1:1.8] to [1:2.5];
wherein the tube (101) comprises a first end (102) for receiving a fluid (200) into the tube (101);
wherein the tube (101) comprises a second end (103) configured to retain a fluid (200) in the tube (101); and,
one or more slits (104) for dispensing a fluid (200) from the tube (101), wherein the slits (104) each form a passage extending from an inner side (105) of the tube (101) to an outer side (106) of the tube; and wherein the slits (104) are configured to reversibly open depending on the pressure of a fluid (200) within the tube (101) so that the fluid (200) is dispensed from the slits (104).

2. The device according to claim 1, wherein the device is designed and configured to dispense a medical fluid exclusively via the slits, wherein preferably a simultaneous dispensation of the fluid takes place via a plurality of slits at different positions along the longitudinal axis (LA) of the tube.

3. The device according to claim 1, wherein the slits are designed and configured to open above a limit pressure of a fluid inside the tube, which is 1 bar (10\hat{ }5 Pa), preferably 1.4 bar, higher than the pressure outside the tube, and to close fluid-tight below this limit pressure.

4. The device according to claim 1, wherein the slits are configured to reversibly open and close via a resilient restoring force of the first material of the tube.

5. The device according to claim 1, wherein the slits each have a slit length (L), and wherein the ratio of the slit length (L) to the inner diameter (ID) of the tube [L:ID] is in the range from [1:2.2] to [1:2.9], and/or the ratio of the slit length L to the outer diameter (AD) of the tube [L:AD] is in the range from [1:3.5] to [1:5.5].

6. The device according to claim 1, wherein the device is designed and configured to change the outer diameter (AD) of the tube by less than 10% when the pressure of a fluid inside the tube increases from 0 to 1 bar (10\hat{ }5 Pa) higher than the pressure outside the tube.

7. The device according to claim 1, wherein the device is designed and configured to change the length of the tube by less than 10%, preferably less than 5%, when the pressure of a fluid inside the tube increases from 0 to 1 bar (10\hat{ }5 Pa) higher than the pressure outside the tube.

8. The device according to claim 1, wherein the tube comprises a plurality of slits, each arranged at a distance (A) from one another on the outer side of the tube, and each having a slit length (L) along the outer side of the tube, wherein the ratio of distance (A) to slit length (L) [A:L] is at least [10:1].

9. The device according to claim 1, wherein the tube comprises a second material (142) which has a higher Shore A hardness than the first material (141), wherein the second material is preferably arranged as a coaxially encircling layer or as a strip parallel to a longitudinal axis (LA) of the tube, wherein the second material further preferably comprises an elastomer, and wherein further preferably the second material is completely embedded in the first material.

10. The device according to claim 9, wherein the second material comprises a radiopaque agent and/or a dye, wherein the radiopaque agent preferably comprises barium sulfate or tungsten, and wherein the dye preferably has an absolute emission maximum in the range of 490 nm to 575 nm.

11. The device according to claim 1, wherein the slits are arranged parallel to a longitudinal axis (LA) of the tube.

12. The device according to claim 1, wherein in a first longitudinal position (301) in the direction of a longitudinal axis (LA) of the tube a first slit (104) is arranged at a first radial position (401), and in a second longitudinal position (302) in the direction of the longitudinal axis of the tube a second slit (104′) is arranged at a second radial position (402), wherein the first radial position (401) forms an angle of approximately 90° to the second radial position (402).

13. The device according to claim 1, wherein the tube comprises a support structure (130) made of metal, wherein the support structure (130) preferably comprises a metal coating, a metal foil, a metal spiral or a metal thread, and wherein the support structure is preferably embedded in the first material (141) or arranged on the inner side (105) of the tube.

14. The device according to claim 1, wherein the first medically acceptable material (141) comprises a polymer, wherein the polymer preferably comprises polyether urethane or ethylene propylene diene rubber.

15. The device according to claim 1, further comprising a fluid connector (212) which is arranged, preferably detachably arranged, at the first end (102) of the tube (101).

16. The device according to claim 15, wherein the fluid connector comprises a Luer-Lock connector (220) and/or a check valve (240).

17. The device according to claim 1, wherein the second end (103) is closed or closable fluid-tightly, preferably by welding, fusing or by means of a plug or screw cap.

18. The device according to claim 1, wherein the slits (104) can be produced or are produced by severing portions of the tube without removing material from the tube.

19. The device according to claim 1, wherein the device is designed and configured to permanently change the length of the tube (101), for example by removing a tube portion at the second end (103) of the tube.

20. A method of treatment, wherein the method comprises administering a medical fluid to a patient using a device according to claim 1.
